(19) United States
(12) Patent Application Publication
(10) Pub. No.: US 2015/0376235 A1
(43) Pub. Date: Dec. 31, 2015

(54) Peptides for the Treatment of Immune Reconstitution Inflammatory Syndrome (IRIS) and Related Diseases

(71) Applicant: Michael Ruff, Potomac, MD (US)

(72) Inventor: Michael Ruff, Potomac, MD (US)

(21) Appl. No.: 14/315,489

(22) Filed: Jun. 26, 2014

(51) Int. Cl. C07K 7/08; C07K 7/06

(52) U.S. Cl. CPC C07K 7/08; C07K 7/06

(57) ABSTRACT

A method of treatment of Immune Reconstitution Inflammatory Syndrome (IRIS) in a patient is disclosed. The method comprises preparing a composition comprising a D peptide and a pharmaceutically acceptable carrier.,said D peptide further comprises the general structure: A-B-C-D-E-F-G-H in which:
A is Ala, or absent,
B is Ser, Thr or absent,
C is Ser, Thr or absent,
D is Ser, Thr, Asn, Glu, Arg, Ile, Leu,
E is Ser, Thr, Asp, Asn,
F is Thr, Ser, Asn, Arg, Gln, Lys, Trp,
G is Tyr, and
H is Thr, Ser, Arg, Gly, and
   All amino acids are the D stereoisomeric configuration. The composition is administered to the patient in a therapeutically effective dose and the composition acts to treat IRIS in the patient.

The present invention relates, broadly, to the treatment or prevention of excessive inflammation in Immune Reconstitution Inflammatory Syndrome (IRIS) and related diseases, whether caused by injury, bacteria, viruses and/or other infective agents, opportunistic infections (which may be consequential to an immunodepressed state, for example resulting from cancer or therapy, particularly cytotoxic drug therapy, monoclonal antibody therapy to suppress immunity, or radiotherapy), autoimmunity, cessation of immunosuppressive treatments, or initiation of antiviral therapies, or otherwise.

### BRIEF DESCRIPTION OF THE DRAWINGS

FIG. 1 illustrates the chemotaxis of human monocytes for several all-D analogues of Peptide T compared to D-ala¹-peptide T-NH₂, “DAPTA”. The amino acid sequences of the analogues are: ASTTTNYT (SEQ ID NO:1) and TTNYT (SEQ ID NO:2).

FIG. 2 illustrates RAP-103 (All-D-TTNYT) (SEQ ID NO:2) potently blocking both MCP-1/CCR2 and MIP-1β/CCR5-elicited chemotaxis of human monocytes.

FIG. 3 illustrates the effects of several all-D amino acid peptides in blocking CCL2 (MCP-1) chemotaxis at low concentration. The amino acid sequences of these peptides are: SSTYR (SEQ ID NO:3), TTSYT (SEQ ID NO:4) and NTSYR (SEQ ID NO:7)

FIG. 4 illustrates eight (8) all-D-pentapeptides, SEQ ID NOS: 3-10, respectively, with unrelated sequences, all inhibiting CCR2 (MCP-1) elicited chemotaxis of human monocytes.

FIG. 5 illustrates all-D-(IDNYT) (SEQ ID NO:6) potently blocking MCP-1/CCR2 elicited adhesion of human monocytes.

FIG. 6 illustrates lipopolysaccharide (LPS) induction TNFα secretion in monocyte-derived iDC is inhibited by RAP-103 (All-D-TTNYT) (SEQ ID NO:2).

In particular embodiments, the invention relates to the prevention or treatment of immune reconstitution inflammatory syndrome (IRIS) reactions which may present spontaneously or due to underlying HIV, multiple sclerosis (MS), or diseases in humans which are associated with acute immune activation that occurs via cytokine, chemokine, and toll-receptor inflammatory pathways in previously immunosuppressed patients who undergo immune reconstitution as a result of cessation of immunosuppressive therapy or use of TNFα antagonists, or who receive immunorestorative therapy, as occurs in HIV with initiation of antiretroviral therapy (ART). Additional examples include transplant patients following withdrawl of immune therapies, MS patients who discontinue natalizumab or other immunomodulating therapies, individuals with tuberculosis or leprosy following initiation of antimicrobial therapy, rapid recovery from neutropenia, women in the post-partum period, and individuals with autoimmune disorders in association with immune modulating therapies. The invention also relates to pharmaceutical compositions useful in such treatment and/or prevention and to certain active peptides per se.

The present invention relates, broadly to the treatment or prevention of excessive inflammation in Immune Reconstitution Inflammatory Syndrome (IRIS) And Related Diseases, whether caused by injury, bacteria, viruses and/or other infective agents, opportunistic infections (which may be consequential to an immunodepressed state, for example resulting from cancer or therapy, particularly cytotoxic drug therapy, monoclonal antibody therapy to suppress immunity, or radiotherapy), autoimmunity, cessation of immunosuppressive treatments, or initiation of antiviral therapies, or otherwise. The present invention relates to compositions and a method for modulating, in particular reducing, an excessive immune response in an animal, such as a human or another mammal.

In one embodiment, the invention relates to compositions and a method for modulating, and in particular reducing, the inflammatory reaction to opportunistic infections in previously immunosuppressed individuals who experience immune reconstitution either spontaneously or as a result of cessation of an immunosuppressive therapy, or institution of an immune-restorative treatment.

The immune reconstitution inflammatory syndrome (IRIS), also called immune restoration disease, occurs in MS patients upon cessation of immunosuppressive therapy. Return of immune competence may cause worsening of symptoms and neurological disease, which can be severe, even fatal. Even in those who survive disabilities typically persist and recovery is partial. An unmet medical need is to prevent or treat IRIS reactions, wherever they may occur.

More specifically, this embodiment of the invention relates to compositions and a method for modulating, and in particular reducing, the secretion of inflammatory cytokines, by modulating adhesion of immune cells to ICAM-receptors, or by blocking chemokine induced migration of monocytes into damaged tissues, blocking inflammogen activation of brain astrocytes or microglia, or preventing release of free radicals and other toxins that can cause bystander death of brain cells leading to disabilities.

The compositions and method of the invention can therefore be used to alter immune responses to specific antigens as well as immune responses caused by disorders of the immune system, such as may occur in auto-immune diseases, allergy, inflammatory bowel disease, cardiovascular disease, multiple sclerosis, HIV and more specifically immune reconstitution inflammatory syndromes (IRIS) with or without any of the described underlying associated illnesses.

In a further embodiment, the method of the invention can further be used in the treatment of HIV, JC virus, and Herpesvirus infections, such as may occur in the brain, and similar disorders of the immune system, as well as to modulate the immune response to sepsis, grafts or transplants. Further embodiments of the invention relate to prophylactic techniques as well as diagnostic techniques using the compositions and/or embodying the methods as described above.

Antiretroviral therapy (ART) initiation in HIV-infected patients leads to recovery of CD4+ T cell numbers and restoration of protective immune responses against a wide variety of pathogens, resulting in reduction in the frequency of opportunistic infections and prolonged survival. However, in a subset of patients, dysregulated immune response after initiation of ART leads to the phenomenon of immune reconstitution inflammatory syndrome (IRIS). The hallmark of the syndrome is paradoxical worsening of an existing infection or disease process or appearance of a new infection/disease process soon after initiation of therapy. The overall incidence of IRIS is dependent on the population studied and the burden of underlying opportunistic infections and has been reported in 10-32 percent of patients starting anti-retroviral therapy (ART).

IRIS reactions are common in multiple sclerosis (MS) patients who discontinue immunosuppressive therapies for their illness, and 100 percent of MS patients who develop progressive multi-focal leukoencephalopathy (PML) and discontinue Tysabri will develp an IRIS reaction. Although a majority of patients with IRIS have a self-limiting disease course the reactions can be severe and life-threatening. The immunopathogenesis of the syndrome appears to be result of unbalanced reconstitution of effector and regulatory T-cells, leading to exuberant inflammatory response in patients receiving ART.

Biomarkers, including interferon-γ (INF-γ), tumour necrosis factor-α (TNFα), C-reactive protein (CRP) and inter leukin (IL)-2, 6 and 7 have been identified. The commonest forms of IRIS are associated with mycobacterial infections, fungi, JC virus, and herpes viruses. Pathway analyses of monocytes isolated from PBMCs of cART/TB-IRIS patients revealed that the majority of the dysregulated genes in TB-IRIS are associated with infection and inflammation (Tran et al., 2014).

Tuberculosis IRIS is the most commonly occurring IRIS syndrome worldwide. It nearly always presents with fever. Other clinical findings may include worsening infiltrates or new pleural effusion on chest X ray, mediastinal and/or peripheral lymphadenopathy, skin or visceral abscesses, arthritis, and osteomyelitis. Typically, the onset of the syndrome occurs 1 to 6 weeks after ART is initiated in an HIV-infected patient who is already on treatment for active tuberculosis. Nineteen percent of HIV-infected patients with active tuberculosis, and undergoing antimycobacterial therapy, who began combination ART developed IRIS reactions, and 50% of these TB IRIS cases required hospitalization. Median duration reactions, and 50% of these TB IRIS cases required hospitalization.

The MS-IRIS syndrome was first reported in a patient with multiple sclerosis in whom progressive multifocal leukoencephalopathy (PML) developed during treatment with natalizumab (Tysabri), a humanized IgG4 monoclonal antibody directed against the alpha-4-subunit of the heterodimeric alpha-4 integrins (ICAMs). By inhibiting these molecular interactions, natalizumab prevents the recruitment and egress of leukocytes into sites of inflammation. The patient subsequently developed an immune-reconstitution inflammatory syndrome (IRIS) three months after discontinuation of natalizumab (Langer-Gould et al., 2005). Other reports indicate IRIS reactions in this population may occur quickly, within 3 weeks, after plasma exchange is used to accelerate clearance of natalizumab (Linda et al., 2009). In MS patients with natalizumab-related PML who were managed by discontinuation of natalizumab and plasmapheresis/immunoadsorption (PLEX/IA), all developed IRIS reactions (Tan, 2011) and up to 38% of MS patients who discontinued immunosuppressive therapy develop IRIS (Miravalle, 2011). Some reactions were clinically severe requiring intensive care and high-dosage steroid treatment, and many only partially recover (Calvi et al., 2014).

There are still no clear guidelines to properly treat IRIS complications in order to reduce the residual disability of patients surviving this treatment complication. The common, though unproven, use of glucocorticoids to treat IRIS may limit JC viral clearance and is not always effective.

Brain biopsy tissue and MRI scans from five MS patients with natalizumab-associated PML were analyzed and their histology compared with non-MS PML. Histology showed an extensive CD8-dominated T cell infiltrate and numerous macrophages within lesions. The monocyte/macrophages and related cells such as brain microglial and dendritic cells in the skin are sentinels of the body's innate immune system which provides initial host defense and response to trauma, injury, toxins, metabolic syndromes, stressors of various types, and of course very significantly, the microbes. Molecularly this signaling occurs via activation of receptors for the alarmins, chemokines, and cytokines, roughly in that order. IRIS pathophysiology therefore results from excessive activation of innate and specific immune pathways and therapies that limit excessive innate signaling pathways will also block specific immunity and may be treatments for IRIS.

D-ala1-peptide T-amide (DAPTA) is derived from the HIV envelope protein (Pert et al., 1986) and is an antagonist of CCR5 (Polianova et al., 2005) and CCR2 (Padi et al., 2012) which blocks monocyte infiltration into injured brain or spinal cord and lowers expression of inflammatory cytokines such as TNFα, Il-1, IL-6, and IL-8 in animals and people (Ruff et al., 2003; Rosi et al., 2005; Padi et al., 2012)

Because DAPTA blocks the actions of several inflammatory receptors, it provides broader receptor antagonism to inhibit multiple innate immune pathways. DAPTA enters the brain and is also thousands of times more potent than other chemokine antagonists that have entered clinical trials. These distinguishing features may afford more effective “coverage” of the pharmacological receptor cluster that mediates inflammation and causes IRIS and so deliver better therapeutic outcome to block development of the IRIS inflammatory cascade.

All compounds disclosed in these specifications are useful for the present invention. The compounds block multiple chemokine receptors implicated in the discussed immune reconstitution inflammatory syndromes (IRIS), with particular relevance to IRIS reactions in MS and HIV, and the lead compound DAPTA, has shown patient benefits with no toxicities.

The lead compound DAPTA was derived from the octapeptide Ala-Ser-Thr-Thr-Thr-Asn-Tyr-Tyr, SEQ ID NO:1. This all-L amino acid octapeptide was called Peptide T because 50% of the amino acid residues are threonines. This peptide has been identified from the V2 subregion of the human immune deficiency virus (HIV) external glycoprotein molecule gp120, specifically near the bridging sheet to the V3 loop, a region which is responsible for virus binding via the CCR5 and related chemokine receptors, such as CCR2, CCR8, CX3CR1 all of which may function as HIV entry receptors. The peptides we here describe are antagonists of multiple HIV entry chemokine receptors. Peptide T is not stable in the body, however its close derivative D-ala1-peptide T-amide, or “DAPTA” has a single D-amino acid in position 1 and a terminal amide (—NH2) group which confers stability in the blood to some proteases. The DAPTA peptide however easily aggregates upon storage in liquid solutions due to defects in manufacturing and an improved process was developed to overcome this limitation. Further modifications of Peptide T and DAPTA have been created that confer oral bioavailability.

The peptides can be used in pharmaceutical compositions and compositions of matter for treating and preventing any disease or condition caused by an organism, compound or immune dysfunction that results in an inflammatory reaction of the immune system.

The peptides or peptide formulations may be used alone or in combination with any other pharmaceutically active compound, such as an anti-infective agent, for example an antibiotic and/or antiviral agent and/or antifungal agent, or another pharmaceutically active compound, such as an antineoplastic agent or an excipient that enhances delivery through nasal or oral routes of administration.

The peptides may be administered orally, bucally, parenterally, topically, rectally, vaginally, by intranasal inhalation spray, by intrapulmonary inhalation or in other ways. In particular, the peptides according to the invention may be formulated for topical use, for inhalation with spray or powder, for injection (for example subcutaneous, intramuscular, intravenous, intra-articular or intra-cisternal injection), for infusion or for oral administration and may be presented in unit dose form in ampoules or tablets or in multidose vials or other containers with an added perservative. The compositions may take such forms as suspensions, solutions, or emulsions or gels in oily or aqueous vehicles, and may contain formulatory agents such as suspending, stabilising and/or dispersing agents. Alternatively, the active ingredient may be in powder and/or lyophilised form for direct administration or for constitution with a suitable vehicle (e.g. sterile, pyrogen-free water, normal saline or 5% dextrose) before use. The pharmaceutical compositions containing peptides(s) may also contain other active ingredients such as antimicrobial agents, or preservatives.

The compositions may contain from 0.001-99% (w/v or, preferably, w/w) of the active material.

The compositions are administered in therapeutically or prophylactic effective does, i.e. 0.05-1000 mg of peptide per day, in particular 5-500 mg per day. Very large doses may be used as the peptide according to the invention is non-toxic. However, normally this is not required. The dose administered daily of course depends on the degree of inflammation and inflammatory response.

For administration by injection or infusion of the compositions, the daily dosage, as employed for treatment of adults of approximately 70 kg of body weight, will often range from 5-500 mg of active material which may be administered in the form of 1 to 4 doses over each day, The invention may be useful in the prevention or treatment of illness or medical conditions, particularly those involving inflammation, such as: viral, bacterial or drug-induced brain inflammation, encephalitis, spontaneously or associated with treatment cessation, as in MS/Tysabri, or initiation, as in HIV/ART; sepsis/septic shock; dermal inflammation; immunosuppressive therapies used to prevent graft rejection, or treatment of cancers or neoplastic diseases, rheumatoid arthritis, or autoimmune conditions.

The invention finds particular use in the prevention or treatment of IRIS associated with MS, HIV and other immunosuppressed states in which immune reconstitution may occur, either spontaneously or due to therapeutic interventions.

More particularly, the invention is useful in treating neurodegenerative IRIS reactions, systemic IRIS reactions, chronic fatigue syndromes, toxic shock syndrome associated with Staphylococcus aureus infection, and host-versus-graft response in transplant patients. Such efficacious results in the use of the above compounds is thought to be due, without being limited to any particular theory, to the immunosuppressive activities of these compounds in both acute and chronic inflammatory states.

### Oral Bioavailability

An unexpected aspect of the present invention is the use of all-D amino-acids in the creation of the bioactive peptides that target chiral molecules, such as cell surface GPCR receptors. A recent review of oral delivery of therapeutic proteins and peptides by Gupta, (2013), indicates that “Despite extensive research efforts, oral delivery of a therapeutic peptide or protein is still a challenge for pharmaceutical industries and researchers. Therefore, because of the short circulatory half-life exhibited by peptides in vivo, they need to be administered frequently resulting in increased cost of treatment and low patient compliance” and in many cases oral delivery is not even possible. Generally, protein and peptide drugs are rapidly denatured or degraded by the low pH environment of the gastric media or the hydrolytic enzymes in the gastrointestinal tract (Ensign et al., 2014). Despite their growing importance and almost 100 years of research, the vast majority of peptide drugs are still only available by injection. “Oral bioavailabilities of peptide and protein drugs are very low mainly because of the stability and permeability barriers of the gastrointestinal tract ” (Smart et al., 2014). Despite tremendous efforts, parenteral delivery still remains the major mode of administration for protein and peptide therapeutics. Other routes such as oral, nasal, pulmonary and buccal are considered more opportunistic rather than routine application. (Patel et al., 2014).

Some examples of all-D amino acid therapeutic peptide antibiotics exist, such as the theta-defensin described by Owen (Owen et al., 2004). Human defensins are cationic peptides that self-associate into dimers and higher-order oligomers. They bind protein toxins, such as anthrax lethal factor (LF), and kill bacteria, including Escherichia coli and Staphylococcus aureus and the theta-defensins can bind to the HIV viral particle to block infection.

The defensin peptide is rich in arginines and its interactions with microbes are electrostatic and disruptive of membranes. That an all-D defensin can produce some neutralization of HIV appears to be a function of the less discriminatory recognition of these cationic peptides for carbohydrates, DNA or lipid moieties of the defensing peptides as evinced their wide activity against toxins, bacteria, fungi, and viruses. The mechanism of action does not involve interaction with a stereospecific receptor moiety but rather charged based binding to a microbe.

This has been explained previously for other antibiotic peptides. Thus the D enantiomers of three naturally occurring antibiotics—cecropin A, magainin 2 amide, and melittin were studied. All of the peptides were potent antibacterial agents against representative Gram-negative and Gram-positive species. The D and L enantiomers of each peptide pair were equally active, within experimental error. It was suggested that the mode of action of these peptides on the membranes of bacteria, erythrocytes, plasmodia, and artificial lipid bilayers may be similar and involves the formation of ion-channel pores spanning the membranes, but without specific interaction with chiral receptors or enzymes. (Wade et al., 1990).

The results showing receptor-mediated effects of short all-D peptides which work via binding to chiral receptors that mediate downstream signaling actions are therefore quite distinct from the non-specific electrostatic binding of, e.g., defensins. Furthermore, in view of the many recent references to the difficulty of creating orally active peptides, these results are unexpected.

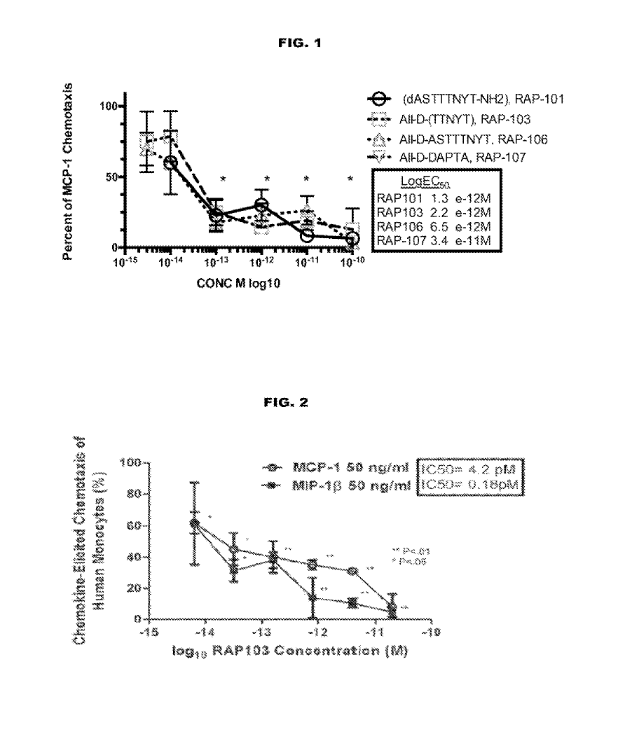

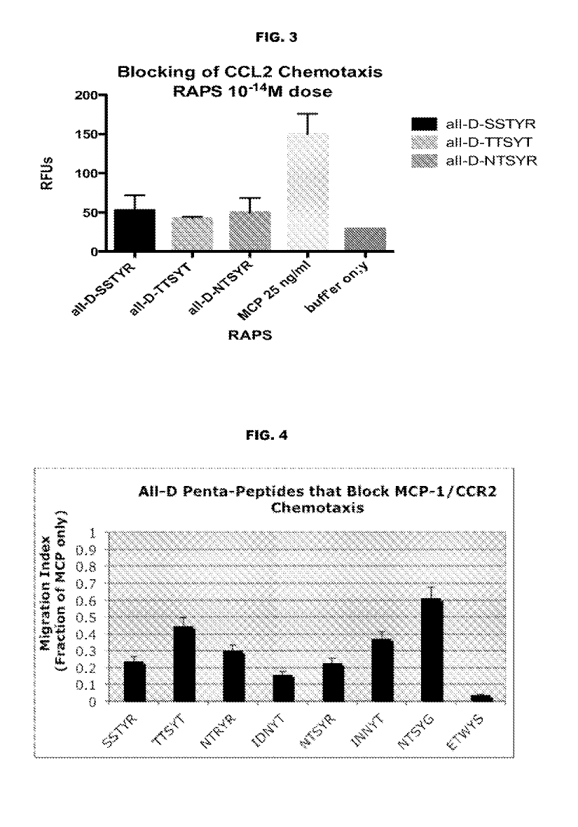

The biopotency of all-D peptides which we here describe is unexpected in view of additional specific previous work, Pert, PNAS, 83:9254,1986, FIGS. 3 and 4, which showed that that D for L substitutions in linear peptide of General Formula 1 (see below), of which the specific example is ASTTTNYT, SEQ ID NO:1, can cause great loss of potency.

Having one D substitution, in the specific position No. 1, (the D-ala) retains potency. Making an additional D substitution, in the specific position No 8 (the D-Thr) results in loss of 99 to 99.9% of the activity. Thus it is shown that introduction of L to D substitutions cannot be made in a general fashion, and that these modifications can, and typically do, destroy biopotency by disrupting the peptide structure required for receptor potency and have been specifically shown to destroy biopotency in the subject peptides.

This point is further made in Brenneman, Drug Dev Res 15:361,1988., with specific reference to the peptide TTNYT, SEQ ID NO:2. See FIG. 2 and Table 1. Upon making the L to D substitution in position 4 (Tyr), the peptide completely loses activity. This directly contradicts Andersen, U.S. Pat. No. 6,265,374 because each of the amino-acids cannot be in the D-form and retain biopotency. The objection is not overcome as Andersen provides no evidence that each amino-acid can be in a D-form.

The notion that an all-D peptide would retain significant receptor potency is furthermore novel in consideration of long the established understanding that such modificatons are not possible as shown in Stewart and Woolley, Nature, 206:619, 1965 who prepared all-D peptides for the receptor-active peptides MSH and bradykinin. For example, from their article, “In contrast to the change of a single residue, the inversion of all the amino-acid residues in a pentapeptide which has hormonal activity of MSH was found to cause loss of hormonal activity . . . ”. Further in this paper the authors stated that because there is as yet no general method for predicting the structural requirements required to make antimetabolites of peptides, we synthesized all-D bradykinin (note 9 amino acids, similar size to the 8 amino acid Formula 1 peptide of Andersen) in an effort to find out whether inversion of all the amino-acids of a peptide may be a generally applicable method for synthesis of peptide antagonists.”

The authors then concluded that “amounts of all-D-bradykinin up to 50,000 times the standard challenge of bradykinin showed neither any inhibition of the response to bradykinin, or any bradykinin-like effect. It would thus seem that inversion of all the amino-acid residues may not be a generally applicable method for formation of antimetabolites of biologically active peptides” (emphasis added).

A detailed study of the peptide TTNYT, SEQ ID NO:2, and L to D substitutions was published in Smith, Drug Dev Res, 1988. Refer to FIG. 3. Introduction of single L to D substitutions in each position 1, 2, 3, 4, results in loss of potency, and all of the D form substitutions are substantially less active (50×) to completely inactive.

As such the use of D-substitutions by Andersen in “each” position has not been reduced to practice. The data shows that in no instance does a D for L amino-acid substitution achieve comparable potency to the all-L form, rather D substitutions result in loss of activity, sometimes complete loss of biopotency in a position dependent fashion.

In contrast to the repeated findings of numerous authors (op. cit. above) the author of the current study discovered, while seeking to construct a negative control, inactive version of linear octapeptide of General Formula 1, that a linear octapeptide of General Formula 1 comprised of all-D-amino-acid substitutions, as well as all-D-amino-acid substitutions of linear pentapeptide analogs of the linear octapeptide of General Formula 1, do retain comparable potency as the all-L or single-D-substituted peptides first described in Pert, 1985. Thus there was little loss of potency, a result unexpected in view of Smith, Drug Dev Res, 1988 and Brenneman, Drug Dev Res 15:361,1988., with specific reference to the peptide TTNYT, SEQ ID NO:2.

An example is provided in FIG. 1. We synthesized three all-D-amino acid peptide analogs of DAPTA, such as “all-D-DAPTA” (RAP-107), all-D-Peptide T (RAP-106), and the shorter pentapeptide that contains the core bioactive moiety of Peptide T (Ruff, 1987).

The results show that three “all-D” peptides, comprised of D, not L, amino acids, retained nearly full potency, at sub-pM concentrations, to antagonize chemokine receptors or block human mononuclear cell binding to integrins and have in vivo benefits in animal models of inflammation (Padi, 2012).

In order to show receptor targets we evaluated the ability of one of the new class of all-D peptides, (all-D[TTNYT], aka RAP-103) (SEQ ID NO:2) to block chemokine chemotaxis caused by the CCR2 and CCR5 receptors. The results are presented in FIG. 2.

FIG. 2 shows all-D[TTNYT], generic name RAP-103, SEQ ID NO:2, is an antagonist of CCR5 and CCR2 human monocyte chemotaxis. MCP-1 is CCL1, and MIP-1β is CCL4, Data are from [Padi, 2012, FIG. 1]. The result shows a further un-anticipated action of this family of peptides related to the HIV V2-region derived Peptide T related to its ability to block CCR2 chemotaxis. Previously an ability of Peptide T and DAPTA to block CCR5 was shown (Redwine, 1999). The peptides described herein therefore are at least dual-chemokine receptor antagonists as they block both CCR2 and CCR5. Dual-chemokine receptor antagonists may have added therapeutic value by blocking multiple inflammatory pathways.

FIG. 2 shows all-D[TTNYT]/RAP-103, SEQ ID NO:2, potently blocking both MCP-1- and MIP-1β-elicited chemotaxis of human monocytes. Monocytes were treated with the indicated doses of RAP-103 for 30 min before chemotaxis against human MCP-1 or MIP-1β (both 50 ng/mL) for 90 min. Data are the average of 2 separate experiments, conducted with triplicate determinations. Data (chemotactic index) are presented as mean±SEM. The IC50 for inhibition of MCP-1 or MIP-1β was generated by a nonlinear inhibition curve fit in GraphPad Prism software, version 5.0. The chemotactic index for MCP-1 without RAP-103 was 2.5-3.5 times over control, whereas for MIP-1β without RAP-103, it was approximately 2 times over control. Data are presented as mean±SEM. *P<0.05, **P<0.01 vs all-D[TTNYT]/RAP-103, SEQ ID NO:2, untreated.

In order to show the generalizability of the all-D-amino acid modifications we synthesized additional examples and tested them for antagonism of chemokines receptors implicated in the subject diseases, FIG. 3

Additional All-D-(Pepntapeptides) from V2 Region Block CCR5/CCR2 Human Monocyte Chemotaxis

In FIG. 3, the effects of RAPs in blocking CCL2 (MCP-1) chemotaxis was illustrated. Compounds were tested at a concentration of 10⁻¹⁴ M. All of the compounds were highly active to antagonize CCL2. Triplicate determinations were performed and results are expressed as the mean plus or minus SEM. The experiment shown is a direct comparison among all RAPs. Statistical analysis was by unpaired t-test, with significance set at the p<0.01 (*) level for difference from CCL2 only chemotaxis.

The results show that all-D-versions of additional HIV gp120 V2-region pentapeptides (SSTYR, TTSYT, NTSYR) (SEQ ID NOS: 3, 4 and 7, respectively) retain potency and are antagonists of chemokine receptors.

We broadened the list of efficacious all-D-peptides to include five more unique examples (NTRYR, IDNYT, IDNYT, NTSYG, ETWYS) (SEQ ID NOS: 5, 6, 8, 9, 10, respectively) of HIV envelope protein derived peptides related to Peptide T that potently block CCR2/CCR5 chemotaxis.

Data on these five examples is shown in FIG. 4. These all-D-pentapeptides inhibit CCR2 (MCP-1) elicited chemotaxis of human monocytes. Purified human monocytes were treated with 20 pM of All-D-pentapeptides for 30 minutes prior to chemotaxis against human MCP-1 (0.6 nM) for 2 hours. The chemotactic index (ratio of migration for CCR2/buffer) for MCP-1 was 3-4. A representative experiment is shown comprising triplicate determinations and is presented as relative fluorescence units, Mean±SEM. The activity of All-D-pentapeptide TTNYT (RAP-103) (SEQ ID NO:2) to block MCP-1 human monocyte chemotaxis has been published, (Padi et al., 2012).

The usefulness of the subject compounds in MS is further suggested by additional actions of the all-D peptides to inhibit β-Integrin-mediated adhesion to human fibronectin. The results are of interest as approved treatments for MS include the immunosupressive monoclonal antibody therapy “Tysabri”, which blocks T cell binding to cellular integrins, and thereby infiltration of inflammatory cells into brains of MS patients. The results suggest further anti-inflammatory mechanisms to block infiltration of T cells or monocytes into brain, useful in treating MS patients.

All-D-(IDNYT) Inhibition of Human Monocyte and THP-1 Cell β-Integrin-Mediated Adhesion to hu-Fibronectin

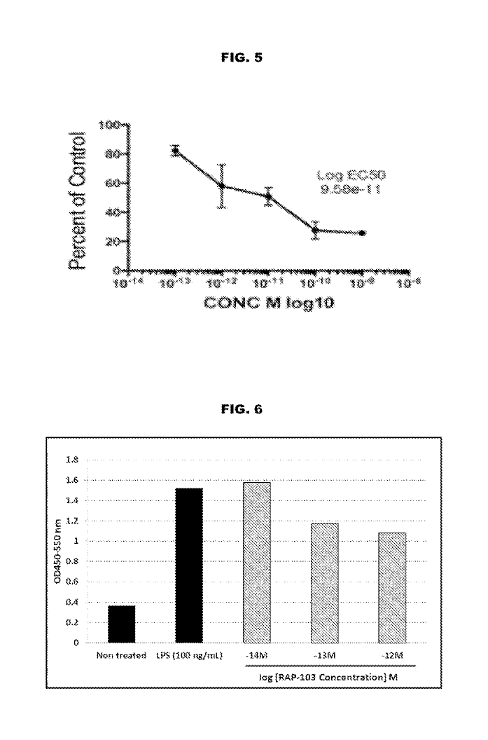

FIG. 5 illustrates all-D-(IDNYT) (SEQ ID NO:6) potently blocking MCP-1 elicited adhesion of human monocytes. Human monocytes were treated with the indicated doses of all-D-(IDNYT) for 10 minutes prior to adherence to the β-integrin human fibronectin. Data is the average of two separate experiments, conducted with triplicate determinations. Data are presented as Mean±SEM of the normalized adherence response from two experiments. The IC₅₀ for inhibition of MCP-1 stimulated adherence was generated using a nonlinear inhibition curve fit in GraphPad Prism Version 5.0.

D-Ala1-Peptide T-Amide (DAPTA/RAP-101) and All-D-[TTNYT] (RAP-103) have Anti-Inflammatory Effects by Lowering Neurotoxic Cytokines in People and Animals.

A further action of the subject peptides relevant to degenerative diseases of inflammation is the ability to decrease the inflammatory cytokines, chemokines, and receptors which underlie disease processes in MS, TSP/HAM and the other inflammatory conditions. Here we show that D-ala1-peptide T-amide (DAPTA, RAP-101), which has only 1 of 8 amino acids in the D-configuration, lowers inflammatory cytokine levels in humans. The effect is shared by the pentapeptide all-D-TTNYT (RAP-103) (SEQ ID NO:2), which was administered by oral gavage, (0.05-1 mg/kg) for 7 days to sciatic nerve injured rats. The specific experimental details are provided in Padi, 2012. Both D-ala1-peptide T-amide and all-D-TTNYT share receptor targets, and biological effects indicating they are analogs that target the same pathological processes. All of the members of the class of HIV gp120, V2 region derived peptides that we describe are therefore expected to share the same actions, benefits, and therapeutic mechanisms, as is expected from structurally related analogs.

TABLE 1
SUMMARY OF INFLAMMATORY BIOMARKER
CHANGES FOR DAPTA (RAP-101) AND ALL-
D-TTNYT (RAP-103) (SEQ ID NO: 2)

| Biomarker | Species | Change | DRUG | Reference |
|---|---|---|---|---|
| IL-1 | Hu | decrease | RAP-101 | Ruff, 2003 |
| IL-6 | Hu | decrease | RAP-101 | Ruff, 2003 |
| IL-8 | Hu | decrease | RAP-101 | Ruff, 2003 |
| TNFα | Hu | decrease | RAP-101 | Ruff, 2003 |
| MCP-1 | Rat | decrease | RAP-103 | unpublished |
| MIP-1α | Rat | decrease | RAP-103 | unpublished |
| TNFα | Rat | decrease | RAP-103 | unpublished |
| CCL2 | Rat | decrease | RAP-103 | unpublished |
| CCL3 | Rat | decrease | RAP-103 | unpublished |
| CCR2 | Rat | decrease | RAP-103 | unpublished |
| CCR5 | Rat | decrease | RAP-103 | unpublished |
| IL-1β | Rat | decrease | RAP-103 | Padi, 2012 |
| IL-6 | Rat | decrease | RAP-103 | Padi, 2012 |

Septic shock is an illustration of a disease involving inflammation. Many of the clinical features of Gram-negative septic shock may be reproduced in animals by the administration of lipopolysaccharide (LPS). The administration of LPS to animals can prompt severe metabolic and physiological changes that can lead to death. Associated with the injection of LPS is the extensive production of TNFα and IL-1β, with their common functional activities such as pyrogenicity, somnogenicity and being mediators of inflammation, have been implicated in the pathology of IRIS reactions, apart from toxic shock and cancer-related cachexia. TNFα has been detected in synovial fluid in patients with both rheumatoid and reactive arthritis and in the serum of patients with rheumatoid arthritis (Saxne et al, 1988, Arthrit. Rheumat. 31, 1041). Raised levels of TNFα have been detected in renal transplant patients during acute rejection episodes (Maury and Teppo 1987, J. Exp. Med. 166, 1132). In animals, TNFα has been shown to be involved in the pathogenesis of graft-versus-host disease in skin and gut following allogenic marrow transplantation.

In view of the effects of the subject peptides, including the all-D-peptide analogs of Peptide T such as all-D-[TTNYT] (RAP-103) (SEQ ID NO:2) to lower TNFα levels in humans and animals, we explored the effect of all-D-[TTNYT] (RAP-103) to block TNFα production from cultured human dendritic cells (iDCs). Such an effect would have benefits in septic shock or other conditions with elevated TNFα levels.

### RAP-103 (All-D-[TTNYT]) Blocks TLR4

FIG. 6 shows that LPS induces TNFα secretion in monocyte-derived iDC which can be blocked by all-D peptide. Cells were incubated for 5 h in medium alone or LPS only at a concentration of 100 ng/mL (black columns), or medium containing LPS plus RAP103 (All-D-TTNYT) (SEQ ID NO:2) (light grey columns). Supernatants were analyzed for secreted TNFα after 5 hours. The bars represent absorbance in an ELISA assay and correspond to TNFα levels.

We tested a high (100 ng/ml) concentration of LPS, nevertheless, all-D[TTNYT]/RAP103 was able to effectively blunt this response at low (pM) concentrations, results consistent with other data showing that the parent compound D-ala1-peptide T-amide/RAP-101 also blocks LPS activation of microglia and NfKb activation (Rosi et al., 2005) and TNFα secretion (Phipps and MacFadden, 1996).

The IC₅₀ for all-D [TTNYT]/RAP103 inhibition of TNFα production was approximately 10⁻¹³ M. The effect of RAP103 to block TLR4 may be allosteric or act via an accessory protein, and may be upstream of chemokine receptor activation since TLR4 signaling typically releases chemokines and cytokines. Treatment uses in lowering TNFα levels are suggested by these in vitro and in vivo (Table 1) data.

The ability to substitute a D for an L amino acid and retain biopotency creates the possibility to make peptides orally deliverable drug compounds to enhance medical treatment. Stability of peptides in biological fluids, such as plasma, or digestive enzymes has limited their utility as drugs. The ability to create all-D peptides that retain potency is an unexpected general method of creating peptides of General Formula 1, and likely many others, which may be stabilized to proteolysis, while retaining biopotency, so a therapeutic may be administered to people via oral dosing or otherwise enjoy enhanced bioavailability in the body.

A pharmacokinetic study of all-D [TTNYT] (RAP-103) (SEQ ID NO:2) following intravenous and oral administration was conducted at a target dose level of 1 mg/kg in the male Sprague Dawley rat. The concentration of RAP-103 in each plasma and brain sample was measured using a suitable LC-MS/MS assay. The assay used was a research grade assay (RGA-1) which was established by assessing the accuracy, precision and the linearity of the method. Plasma concentrations generated were used to evaluate the pharmacokinetic parameters of all-D-TTNYT (RAP-103).

The dose formulation was administered intravenously to multiple animals as a slow bolus over ca 30 s via the tail vein and to a different group of animals orally via gastric gavage at a target dose volume of 1 mL/kg, to achieve a target dose level of 1 mg/kg. The dose volume administered was calculated according to the bodyweight of the animal on the day of dosing. The weight of administered dose was recorded. All dose administrations were well tolerated and no adverse effects from the treatments were observed. The results of the study are in Table 2. Results expressed as ng/mL

TABLE 2
CONCENTRATION OF ALL-D-TTNYT (RAP-103) IN
MALE RAT PLASMA FOLLOWING ORAL ADMINIS-
TRATION AT A TARGET DOSE LEVEL OF 1 MG/KG.
Results expressed as ng/mL

| Nominal Time | Animal | Animal | Animal |
|---|---|---|---|
| (h) | 004M | 005M | 006M |
| 0.083 | <LLOQ | <LLOQ | <LLOQ |
| 0.25 | <LLOQ | 6.42 | 9.33 |
| 0.5 | 10.2 | 7.13 | 9.85 |
| 1 | <LLOQ | <LLOQ | <LLOQ |
| 2 | 12 | 7.06 | <LLOQ |
| 4 | 9.09 | <LLOQ | <LLOQ |
| 8 | 6.67 | <LLOQ | <LLOQ |
| 12 | <LLOQ | <LLOQ | <LLOQ |

LLOQ < 5.00 ng/mL

The results show that as quickly as can be determined, 5 minutes (0.083 hrs) after dosing, all-D [TTNYT] (RAP-103) (SEQ ID NO:2) is detected in plasma, and continues to be detected at 1 hr. Peak levels occur at 10-15 minutes.

The biological relevance is shown in the publication by Padi et al., (Padi, 2012) that shows oral administration of all-D [TTNYT] (0.05-1 mg/kg) for 7 days fully prevents mechanical allodynia and inhibits the development of thermal hyperalgesia after partial ligation of the sciatic nerve in rats. Administered from days 8 to 12, all-D [TTNYT] (0.2-1 mg/kg) reverses already established hypersensitivity. RAP-103 relieves behavioral hypersensitivity through either or both CCR2 and CCR5 blockade. Moreover, all-D [TTNYT] is able to reduce spinal microglial activation and monocyte infiltration, and to inhibit inflammatory responses evoked by peripheral nerve injury that cause chronic pain. The findings indicate that targeting CCR2/CCR5 should provide greater efficacy than targeting CCR2 or CCR5 alone, and that the dual CCR2/CCR5 antagonist all-D [TTNYT] has the potential for broad clinical use in treatment of IRIS reactions.

In this way the peptides can be used in pharmaceutical compositions and compositions of matter for treating and preventing any disease or condition caused by an organism, compound or immune dysfunction that results in an inflammatory reaction of the immune system. The peptides or peptide formulations may be used alone or in combination with any other pharmaceutically active compound, such as an anti-infective agent, for example an antibiotic and/or antiviral agent and/or antifungal agent, or another pharmaceutically active compound, such as an antineoplastic agent.

The peptides may be administered orally, bucally, parenterally, topically, rectally, vaginally, by intranasal inhalation spray, by intrapulmonary inhalation or in other ways. For inflammation in the eye, such as uveitis, the peptides may be administered as eye drops, or systemically. In particular, the peptides according to the invention may be formulated for topical use, for inhalation with spray or powder, for injection (for example subcutaneous, intramuscular, intravenous, intra-articular or intra-cisternal injection), for infusion or for oral administration and may be presented in unit dose form in ampoules or tablets or in multidose vials or other containers with an added perservative. The compositions may take such forms as suspensions, solutions, or emulsions or gels in oily or aqueous vehicles, and may contain formulatory agents such as suspending, stabilising and/or dispersing agents. Alternatively, the active ingredient may be in powder and/or lyophilised form for direct administration or for constitution with a suitable vehicle (e.g. sterile, pyrogen-free water, normal saline or dextrose or mannose) before use. The pharmaceutical compositions co taining peptides(s) may also contain other active ingredients such as antimicrobial agents, or preservatives.

### LITERATURE CITED

Brenneman, D. E., J. M. Buzy, M. R. Ruff, and C. B. Pert. 1988. Peptide T sequences prevent neuronal cell death produced by the envelope protein (gp120) of the human immunodeficiency virus. Drug Devel Res. 15:361-369
Calvi, A., M. De Riz, A. M. Pietroboni, L. Ghezzi, V. Maltese, A. Arighi, G. G. Fumagalli, F. Jacini, C. Donelli, G. Comi, D. Galimberti, and E. Scarpini. 2014. Partial recovery after severe immune reconstitution inflammatory syndrome in a multiple sclerosis patient with progressive multifocal leukoencephalopathy. Immunotherapy. 6:23-28. doi:10.2217/imt.13.155.
Ensign, L. M., Cone, R., and Hanes, J. 2014. Nanoparticle-based drug delivery to the vagina: A review. J Control Release. 10.1016/j.jconre1.2014.04.033.
Giacomini, P. S., A. Rozenberg, I. Metz, D. Araujo, N. Arbour, and A. Bar-Or. 2014. Maraviroc and JC virus-associated immune reconstitution inflammatory syndrome. N Engl J Med. 370:486-488. doi:10.1056/NEJMc1304828.
Gupta, S., A. Jain, M. Chakraborty, J. K. Sahni, J. Ali, and S. Dang. 2013. Oral delivery of therapeutic proteins and peptides: a review on recent developments. Drug Deliv. 20:237-246.
Langer-Gould, A., S. W. Atlas, A. J. Green, A. W. Bollen, and D. Pelletier. 2005. Progressive multifocal leukoencephalopathy in a patient treated with natalizumab. N Engl J Med. 353:375-381. doi:10.1056/NEJMoa051847.
Linda, H., A. von Heijne, E. O. Major, C. Ryschkewitsch, J. Berg, T. Olsson, and C. Martin. 2009. Progressive multifocal leukoencephalopathy after natalizumab monotherapy. N Engl J Med. 361:1081-1087. doi:10.1056/NEJMoa0810316.
Martin-Blondel, G., L. Cuzin, P. Delobel, V. Cuvinciuc, H. Dumas, M. Alvarez, P. Massip, and B. Marchou. 2009. Is maraviroc beneficial in paradoxical progressive multifocal leukoencephalopathy-immune reconstitution inflammatory syndrome management? AIDS. 23:2545-2546. doi:10.1097/QAD.0b013e32833365f4.
Owen, S. M., D. Rudolph, W. Wang, A. M. Cole, M. A. Sherman, A. J. Waring, R. I. Lehrer, and R. B. Lal. 2004. A theta-defensin composed exclusively of D-amino acids is active against HIV-1. J Pept Res. 63:469-476.
Padi, S. S., X. Q. Shi, Y. Q. Zhao, M. R. Ruff, N. Baichoo, C. B. Pert, and J. Zhang. 2012. Attenuation of rodent neuropathic pain by an orally active peptide, RAP-103, which potently blocks CCR2- and CCR5-mediated monocyte chemotaxis and inflammation. Pain. 153:95-106. doi:10.1016/j.pain.2011.09.022.
Patel, A., K. Cholkar, and A. K. Mitra. 2014. Recent developments in protein and peptide parenteral delivery approaches. Ther Deliv. 5:337-365. doi:10.4155/tde.14.5.
Pert, C. B., J. M. Hill, M. R. Ruff, R. M. Berman, W. G. Robey, L. O. Arthur, F. W. Ruscetti, and W. L. Farrar. 1986. Octapeptides deduced from the neuropeptide receptor-like pattern of antigen T4 in brain potently inhibit human immunodeficiency virus receptor binding and T-cell infectivity. Proc Natl Acad Sci USA. 83:9254-928.
Phipps, D. J., and D. K. MacFadden. 1996 Inhibition of tumour necrosis factor-alpha explains inhibition of HIV replication by peptide T. AIDS. 10:919-920
Polianova, M. T., F. W. Ruscetti, C. B. Pert, and M. R. Ruff. 2005. Chemokine receptor-5 (CCR5) is a receptor for the HIV entry inhibitor peptide T (DAPTA). Antiviral Res. 67:83-92
Redwine, L. S., C. B. Pert, J. D. Rone, R. Nixon, M. Vance, B. Sandler, M. D. Lumpkin, D. J. Dieter, and M. R. Ruff. 1999
Rosi, S., C. B. Pert, M. R. Ruff, K. Gann-Gramling, and G. L. Wenk. 2005. Chemokine receptor 5 antagonist D-Ala-peptide T-amide reduces microglia and astrocyte activation within the hippocampus in a neuroinflammatory rat model of Alzheimer's disease. Neuroscience. 134:671-676
Ruff, M. R., P. L. Hallberg, J. M. Hill, and C. B. Pert. 1987. Peptide T[4-8] is core HIV envelope sequence required for CD4 receptor attachment [letter]. Lancet. 2:751
Ruff, M. R., M. Polianova, C. B. Pert, and F. W. Ruscetti. 2003. Update on D-Ala-Peptide T-Amide (DAPTA): A Viral Entry Inhibitor that Blocks CCR5 Chemokine Receptors. Curr HIV Res. 1:51-67
Smart, A. L., S. Gaisford, and A. W. Basit. 2014. Oral peptide and protein delivery: intestinal obstacles and commercial prospects. Expert Opin Drug Deliv. 1-13.
Smith, C. C., P. L. Hallberg, P. Sacerdote, P. Williams, E. Sternberg, B. Martin, C. Pert, and M. R. Ruff. 1988. Tritiated D-ala1-peptide T binding: A pharmacologic basis for the design of drugs which inhibit HIV receptor binding. Drug Devel Res. 15:371-379
Stewart, J. M., and D. W. Woolley. 1965. All-D-bradykinin and the problem of peptide antimetabolites. Nature. 206:619-620
Tran, H. T., R. Van den Bergh, T. N. Vu, K. Laukens, W. Worodria, M. M. Loembe, R. Colebunders, L. Kestens, P. De Baetselier, and G. Raes. 2014. The role of monocytes in the development of Tuberculosis-associated Immune Reconstitution Inflammatory Syndrome. Immunobiology. 219:37-44.
Wade, D., A. Boman, B. Wahlin, C. M. Drain, D. Andreu, H. G. Boman, and R. B. Merrifield. 1990. All-D amino acid-containing channel-forming antibiotic peptides. Proc Natl Acad Sci USA. 87:4761-4765
Calvi, A., M. De Riz, A. M. Pietroboni, L. Ghezzi, V. Maltese, A. Arighi, G. G. Fumagalli, F. Jacini, C. Donelli, G. Comi, D. Galimberti, and E. Scarpini. 2014. Partial recovery after severe immune reconstitution inflammatory syndrome in a multiple sclerosis patient with progressive multifocal leukoencephalopathy. Immunotherapy. 6:23-28. doi:10.2217/imt.13.155.
Ensign, L. M., R. Cone, and J. Hanes. 2014. Nanoparticle-based drug delivery to the vagina: A review. J Control Release. 10.1016/j.jconre1.2014.04.033.
Langer-Gould, A., S. W. Atlas, A. J. Green, A. W. Bollen, and D. Pelletier. 2005. Progressive multifocal leukoencephalopathy in a patient treated with natalizumab. N Engl J Med. 353:375-381. doi:10.1056/NEJMoa051847.
Linda, H., A. von Heijne, E. O. Major, C. Ryschkewitsch, J. Berg, T. Olsson, and C. Martin. 2009. Progressive multifocal leukoencephalopathy after natalizumab monotherapy. N Engl J Med. 361:1081-1087. doi:10.1056/NEJMoa0810316.
Owen, S. M., D. Rudolph, W. Wang, A. M. Cole, M. A. Sherman, A. J. Waring, R. I. Lehrer, and R. B. Lal. 2004. A theta-defensin composed exclusively of D-amino acids is active against HIV-1. J Pept Res. 63:469-476. doi:10.1111/j.1399-3011.2004.00155.x.
Padi, S. S., X. Q. Shi, Y. Q. Zhao, M. R. Ruff, N. Baichoo, C. B. Pert, and J. Zhang. 2012. Attenuation of rodent neuropathic pain by an orally active peptide, RAP-103, which potently blocks CCR2- and CCR5-mediated monocyte chemotaxis and inflammation. Pain. 153:95-106. doi:10.1016/j.pain.2011.09.022.
Patel, A., K. Cholkar, and A. K. Mitra. 2014. Recent developments in protein and peptide parenteral delivery approaches. Ther Deliv. 5:337-365. doi:10.4155/tde.14.5.
Pert, C. B., J. M. Hill, M. R. Ruff, R. M. Berman, W. G. Robey, L. O. Arthur, F. W. Ruscetti, and W. L. Farrar. 1986. Octapeptides deduced from the neuropeptide receptor-like pattern of antigen T4 in brain potently inhibit human immunodeficiency virus receptor binding and T-cell infectivity. Proc Natl Acad Sci USA. 83:9254-928.
Phipps, D. J., and D. K. MacFadden. 1996 Inhibition of tumour necrosis factor-alpha explains inhibition of HIV replication by peptide T. AIDS. 10:919-920
Polianova, M. T., F. W. Ruscetti, C. B. Pert, and M. R. Ruff. 2005. Chemokine receptor-5 (CCR5) is a receptor for the HIV entry inhibitor peptide T (DAPTA). Antiviral Res. 67:83-92
Rosi, S., C. B. Pert, M. R. Ruff, K. Gann-Gramling, and G. L. Wenk. 2005. Chemokine receptor 5 antagonist D-Ala-peptide T-amide reduces microglia and astrocyte activation within the hippocampus in a neuroinflammatory rat model of Alzheimer's disease. Neuroscience. 134:671-676
Ruff, M. R., M. Polianova, C. B. Pert, and F. W. Ruscetti. 2003. Update on D-Ala-Peptide T-Amide (DAPTA): A Viral Entry Inhibitor that Blocks CCR5 Chemokine Receptors. Curr HIV Res. 1:51-67
Smart, A. L., S. Gaisford, and A. W. Basit. 2014. Oral peptide and protein delivery: intestinal obstacles and commercial prospects. Expert Opin Drug Deliv. 1-13. doi:10.1517/17425247.2014.917077.
Tran, H. T., R. Van den Bergh, T. N. Vu, K. Laukens, W. Worodria, M. M. Loembe, R. Colebunders, L. Kestens, P. De Baetselier, and G. Raes. 2014. The role of monocytes in the development of Tuberculosis-associated Immune Reconstitution Inflammatory Syndrome. Immunobiology. 219:37-44. doi:10.1016/j.imbio.2013.07.004.
Wade, D., A. Boman, B. Wahlin, C. M. Drain, D. Andreu, H. G. Boman, and R. B. Merrifield. 1990. All-D amino acid-containing channel-forming antibiotic peptides. Proc Natl Acad Sci USA. 87:4761-4765

What is claimed is:

1. A method of treatment of Immune Reconstitution Inflammatory Syndrome (IRIS) in a patient comprising the steps of:
preparing a composition comprising a D peptide and a pharmaceutically acceptable carrier,
said D peptide further comprises eight contiguous amino acids having the general structure: A-B-C-D-E-F-G-H in which:
A is Ala, or absent,
B is Ser, Thr or absent,
C is Ser, Thr or absent,
D is Ser, Thr, Asn, Glu, Arg, Ile, Leu,
E is Ser, Thr, Asp, Asn,
F is Thr, Ser, Asn, Arg, Gln, Lys, Trp,
G is Tyr, and
H is Thr, Ser, Arg, Gly, and
wherein all amino acids are the D stereoisomeric configuration, and administering said composition to the patient in a therapeutically effective dose, wherein said composition acts to treat IRIS in the patient.

2. The method as defined in claim 1 wherein IRIS is further characterized by brain function loss.

3. The method as defined in claim 2 wherein said brain function loss is due to a condition selected from the group consisting of: Human Immunodeficiency Virus (HIV) infection, cancer, uveitis, rheumatoid arthritis, immunosuppression, multiple sclerosis (MS) or Progressive Multi-focal Leukoencephalopathy (PML).

4. The method as defined in claim 1 wherein said administering said composition to the patient is selected from the group consisting of administrating: orally, bucally, parenterally, topically, rectally, vaginally, by intranasal inhalation spray, by intrapulmonary inhalation.

5. The method as defined in claim 1 further comprising,
said D peptide is at most twenty (20) D amino acid residues in length and contains five contiguous D amino acid residues that have a sequence selected from the group consisting of:
(SEQ ID NO: 2)
Thr Thr Asn Tyr Thr,
(SEQ ID NO: 3)
Ser Ser Thr Tyr Arg,
(SEQ ID NO: 4)
Thr Thr Ser Tyr Thr,
(SEQ ID NO: 5)
Asn Thr Arg Tyr Arg,
(SEQ ID NO: 6)
Ile Asp Asn Tyr Thr,
(SEQ ID NO: 7)
Asn Thr Ser Tyr Arg,
(SEQ ID NO: 8)
Ile Asn Asn Tyr Thr,
(SEQ ID NO: 9)
Asn Thr Ser Tyr Gly,
(SEQ ID NO: 10)
Glu Thr Trp Tyr Ser.

6. The method as defined in claim 5 further comprising, said D peptide derivative is at most twelve (12) D amino acid residues in length.

7. The method as defined in claim 5 further comprising, said D peptide derivative is at most eight (8) D amino acid residues in length.

8. The method as defined in claim 5 further comprising, said D peptide is five (5) D amino acid residues in length.


SEQUENCE LISTING (10 sequences)

SEQ ID NO: 1 - protein, 8 aa, Human immunodeficiency virus
ASTTTNYT

SEQ ID NO: 2 - protein, 5 aa, Human immunodeficiency virus
TTNYT

SEQ ID NO: 3 - protein, 5 aa, Human immunodeficiency virus
SSTYR

SEQ ID NO: 4 - protein, 5 aa, Human immunodeficiency virus
TTSYT

SEQ ID NO: 5 - protein, 5 aa, Human immunodeficiency virus
NTRYR

SEQ ID NO: 6 - protein, 5 aa, Human immunodeficiency virus
IDNYT

SEQ ID NO: 7 - protein, 5 aa, Human immunodeficiency virus
NTSYR

SEQ ID NO: 8 - protein, 5 aa, Human immunodeficiency virus
INNYT

SEQ ID NO: 9 - protein, 5 aa, Human immunodeficiency virus
NTSYG

SEQ ID NO: 10 - protein, 5 aa, Human immunodeficiency virus
ETWYS